(19) United States
(12) Patent Application Publication
(10) Pub. No.: US 2022/0016281 A1
(43) Pub. Date: Jan. 20, 2022

(54) PHYSICAL USER INTERFACE SANITATION SYSTEM AND METHOD

(71) Applicant: William W. EIGNER, San Diego, CA (US)

(72) Inventor: William W. EIGNER, San Diego, CA (US)

(21) Appl. No.: 17/312,379

(22) PCT Filed: Dec. 11, 2019
(86) PCT No.: PCT/US2019/065812

§ 371 Date: Jun. 9, 2021

Related U.S. Application Data
(60) Provisional application No. 62/778,792, filed on Dec. 12, 2018.

(51) Int. Cl. A61L 2/10; A61L 2/24

(52) U.S. Cl. CPC A61L 2/10; A61L 2/24; A61L 2202/16; A61L 2202/14; A61L 2202/11

(57) ABSTRACT

A touchscreen device sanitation system and method is provided. A touchscreen device is configured with an illumination system that illuminates an external surface of the touchscreen device with ultraviolet light that kills or inactivates disease agents present on the external surface of the touchscreen device. The illumination system may illuminate the external surface of the touchscreen device indirectly from a position within the touchscreen device or directly from a position above the touchscreen device.

### BACKGROUND

### Field of the Invention

The present invention generally relates to user interfaces, especially those having a touchscreen display and more particularly relates to sanitizing physical user interfaces that facilitate the spread of germs, viruses, and bacteria and other disease agents.

### Related Art

Physical user interfaces are significantly increasing with the fast adoption of publicly accessible keypads and publicly accessible touchscreen devices at points of sale and points of information and even private keypads and devices that can be but are not necessarily shared with others. As used herein, physical user interfaces, whether accessed on a public device, private device, or personal device, are collectively referred to as “touchscreen” interfaces or devices. A significant disadvantage of such touchscreen interfaces is that they facilitate the spread of disease agents such as gut bacterial, fecal bacteria, staphylococcus, listeria, klebsiella, and proteus bacteria, all of which can cause illness in humans. Therefore, what is needed is a system and method that overcomes or at least reduces these significant problems found in the conventional systems as described above.

### SUMMARY

To address the significant problems with physical user interfaces described above, disclosed herein are systems and methods for sanitization of physical user interfaces to reduce the spread of bacteria, viruses, microorganisms and/or other disease agents that may cause illness in humans.

In an embodiment, a touchscreen device is configured with an illumination system that illuminates an external surface of the touchscreen device with various forms of energy (e.g., light waves, sound waves, heat, etc.) that kills or inactivates disease agents present on the external surface of the touchscreen device. The illumination system may illuminate the external surface of the touchscreen device indirectly from a position within the touchscreen device or directly from a position near or above the touchscreen device.

Other features and advantages of the present invention will become more readily apparent to those of ordinary skill in the art after reviewing the following detailed description and accompanying drawings.

### BRIEF DESCRIPTION OF THE DRAWINGS

The structure and operation of the present invention will be understood from a review of the following detailed description and the accompanying drawings in which like reference numerals refer to like parts and in which:

FIG. 1A is a block diagram illustrating an example personal touchscreen device according to an embodiment of the invention;

FIG. 1B is a block diagram illustrating an example public touchscreen device according to an embodiment of the invention;

FIG. 2A is a block diagram illustrating an example application of a personal touchscreen device sanitation of a public touchscreen device according to an embodiment of the invention;

FIG. 2B is a block diagram illustrating an example application of a personal touchscreen device sanitation of a public physical user interface device according to an embodiment of the invention;

FIG. 3A is a block diagram illustrating an example personal touchscreen device according to an embodiment of the invention;

FIG. 3B is a block diagram illustrating an example application of a personal touchscreen device sanitation of a public touchscreen device according to an embodiment of the invention;

FIG. 3C is a block diagram illustrating an example application of a personal touchscreen device or wand sanitation of a public physical user interface device according to an embodiment of the invention;

FIG. 4 is a block diagram illustrating an example public touchscreen device with stadium sanitation according to an embodiment of the invention;

FIG. 5 is a block diagram illustrating an example touchscreen device according to an embodiment of the invention;

FIG. 6 is a flow diagram illustrating an example process for automated sanitation of a touchscreen device according to an embodiment of the invention;

FIG. 7A-7C are block diagrams illustrating example point of sale touchscreen devices according to embodiments of the invention; and

FIG. 8 is a block diagram illustrating an example wired or wireless processor enabled device that may be used in connection with various embodiments described herein.

### DETAILED DESCRIPTION

Embodiments disclosed herein provide for systems and methods to disrupt disease agents present on a physical user interface. For example, one method disclosed herein allows for a personal or public touchscreen device to automatically illuminate an external surface of a touchscreen user interface with one or more forms of constant or intermittent energy (e.g., light waves, sound waves, heat, etc.) to substantially decrease the amount of disease agents present or active on the external surface of the touchscreen user interface. The term “disrupt disease agents” as used herein refers to the killing or inactivating microorganisms that cause sickness or disease in humans. The term “sanitize” may also be used herein. These terms do not contemplate complete eradication of disease agents, but instead are meant to convey that the disease agents are reduced or substantially reduced or inactivated in order to correspondingly reduce the likelihood of sickness or disease being communicated from the surface of a physical user interface to a human interacting with the physical user interface.

After reading this description it will become apparent to one skilled in the art how to implement the invention in various alternative embodiments and alternative applications. However, although various embodiments of the present invention will be described herein, it is understood that these embodiments are presented by way of example only, and not limitation. As such, this detailed description of various alternative embodiments should not be construed to limit the scope or breadth of the present invention as set forth in the appended claims.

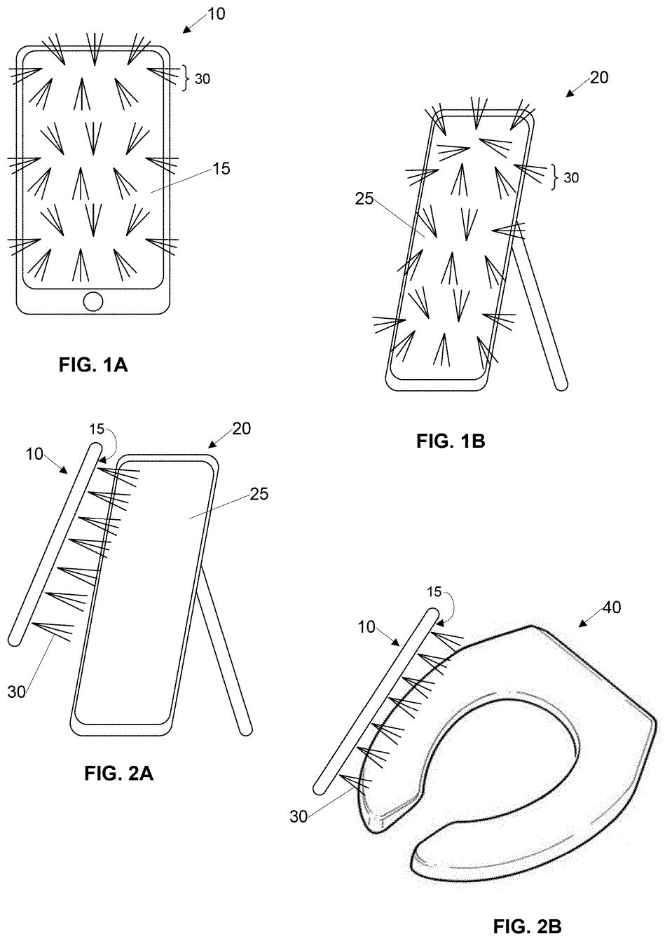

FIG. 1A is a block diagram illustrating an example personal touchscreen device 10 according to an embodiment of the invention. In the illustrated embodiment, the device 10 has a touchscreen user interface 15 that is illuminated from within the device 10. The illumination system within the device 10 is configured to illuminate (and thereby sanitize), from within, the touchscreen user interface 15 and specifically an external surface of the touchscreen user interface 15. The illumination 30 comprises energy at one or more wavelengths and/or sequences of wavelengths that disrupts disease agents. The wavelength may be within the electromagnetic spectrum or the sound spectrum. For example, the illumination 30 may be within the ultraviolet (“UV”) portion of the electromagnetic spectrum substantially between 100 nm and 400 nm. In an embodiment, the illumination 30 may be within the short wave UV spectrum (e.g., UV-C) substantially between 200 nm and 280 nm. In an embodiment, the illumination 30 may be at 254 nm. In an alternative embodiment, the illumination 30 may be within the infrared spectrum. In an embodiment, the energy may be delivered in a constant flood or in pulses. Advantageously, the delivery of the energy may be altered to increase effectiveness of disrupting disease agents. For example, in an embodiment the illumination 30 may include interleaved portions at different wavelengths.

FIG. 1B is a block diagram illustrating an example public touchscreen device 20 according to an embodiment of the invention. In the illustrated embodiment, the device 20 has a touchscreen user interface 25 that is illuminated from within the device 20. The illumination system within the device 20 is configured to illuminate (and thereby sanitize), from within, the touchscreen user interface 25 and specifically an external surface of the touchscreen user interface 25. The illumination 30 comprises energy at one or more wavelengths and/or sequences of wavelengths within the electromagnetic spectrum that disrupts disease agents. For example, the illumination 30 may be within the ultraviolet spectrum substantially between 100 nm and 400 nm. In an embodiment, the illumination 30 may be within the short wave UV spectrum (e.g., UV-C) substantially between 200 nm and 280 nm. In an embodiment, the illumination 30 may be at 254 nm. In an alternative embodiment, the illumination 30 may be within the infrared spectrum.

FIG. 2A is a block diagram illustrating an example application of a personal touchscreen device 10 sanitation of a public touchscreen device 20 according to an embodiment of the invention. In the illustrated embodiment, the personal touchscreen device 10 illuminates (and thereby sanitizes) its own touchscreen user interface 15 and the personal touchscreen device 10 is positioned to cause illumination 30 from its touchscreen user interface 15 to illuminate (and thereby sanitize) at least a portion of an external surface of the touchscreen user interface 25 of the public touchscreen device 20.

FIG. 2B is a block diagram illustrating an example application of a personal touchscreen device 10 sanitation of a public physical user interface device 40 according to an embodiment of the invention. In the illustrated embodiment, the personal touchscreen device 10 illuminates (and thereby sanitizes) its own touchscreen user interface 15 and the personal touchscreen device 10 is positioned to cause illumination 30 from its touchscreen user interface 15 to illuminate (and thereby sanitize) at least a portion of an external surface of the public physical user interface device 40.

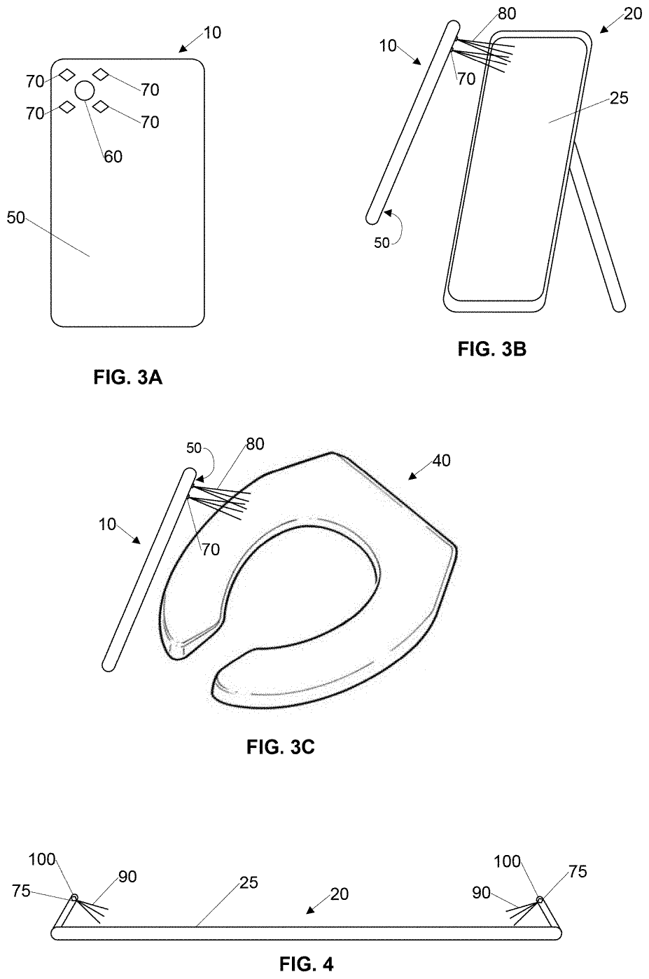

FIG. 3A is a block diagram illustrating an example personal touchscreen device 10 according to an embodiment of the invention. In the illustrated embodiment, the personal touchscreen device 10 comprises a camera system 60 that includes one or more illumination sources 70, for example, positioned on an external surface 50 of the personal touchscreen device 10. The one or more illumination sources 70 are configured to illuminate at least a portion of a field of view of the camera system 60. The illumination 30 comprises energy at one or more wavelengths and/or sequences of wavelengths within the electromagnetic spectrum that disrupts disease agents. For example, the illumination 30 may be within the ultraviolet spectrum substantially between 100 nm and 400 nm. In an embodiment, the illumination 30 may be within the short wave UV spectrum (e.g., UV-C) substantially between 200 nm and 280 nm. In an embodiment, the illumination 30 may be at 254 nm. In an alternative embodiment, the illumination 30 may be within the infrared spectrum.

FIG. 3B is a block diagram illustrating an example application of a personal touchscreen device 10 sanitation of a public touchscreen device 20 according to an embodiment of the invention. In the illustrated embodiment, the personal touchscreen device 10 illuminates one or more illumination sources 70 positioned on an external surface 50 of the personal touchscreen device 10 to cause illumination 80 to illuminate (and thereby sanitize) at least a portion of an external surface of the touchscreen user interface 25 of the public touchscreen device 20. A user of the personal touchscreen device 10 can manipulate the personal touchscreen device 10 to cause more of the external surface of the touchscreen user interface 25 of the public touchscreen device 20 to be illuminated and thereby sanitized.

FIG. 3C is a block diagram illustrating an example application of a personal touchscreen device 10 sanitation of a public physical user interface device 40 according to an embodiment of the invention. In the illustrated embodiment, the personal touchscreen device 10 illuminates one or more illumination sources 70 positioned on an external surface 50 of the personal touchscreen device 10 to cause illumination 80 to illuminate (and thereby sanitize) at least a portion of an external surface of the public physical user interface device 40. A user of the personal touchscreen device 10 can manipulate the personal touchscreen device 10 to cause more of the external surface of the public physical user interface device 40 to be illuminated and thereby sanitized.

FIG. 4 is a block diagram illustrating an example public touchscreen device 20 with stadium sanitation according to an embodiment of the invention. In the illustrated embodiment, one or more risers 100 are positioned above an external surface of the touchscreen user interface 25 of the public touchscreen device 20. The one or more risers 100 support one or more illumination sources 75. The one or more illumination sources 75 are each configured to illuminate at least a portion of the external surface of the touchscreen user interface 25 of the public touchscreen device 20. The illumination 90 comprises energy at one or more wavelengths and/or sequences of wavelengths within the electromagnetic spectrum that disrupts disease agents. For example, the illumination 90 may be within the ultraviolet spectrum substantially between 100 nm and 400 nm. In an embodiment, the illumination 90 may be within the short wave UV spectrum (e.g., UV-C) substantially between 200 nm and 280 nm. In an embodiment, the illumination 90 may be at 254 nm. In an alternative embodiment, the illumination 90 may be within the infrared spectrum.

In one embodiment, the one or more illumination sources 75 may be controlled by a processor within the public touchscreen device 20. In an alternative embodiment, the one or more illumination sources 75 may be controlled by a processor outside of and separate from the public touchscreen device 20. For example, the one or more risers 100 and the one or more illumination sources 75 may be an after-market product that is applied to the public touchscreen device 20.

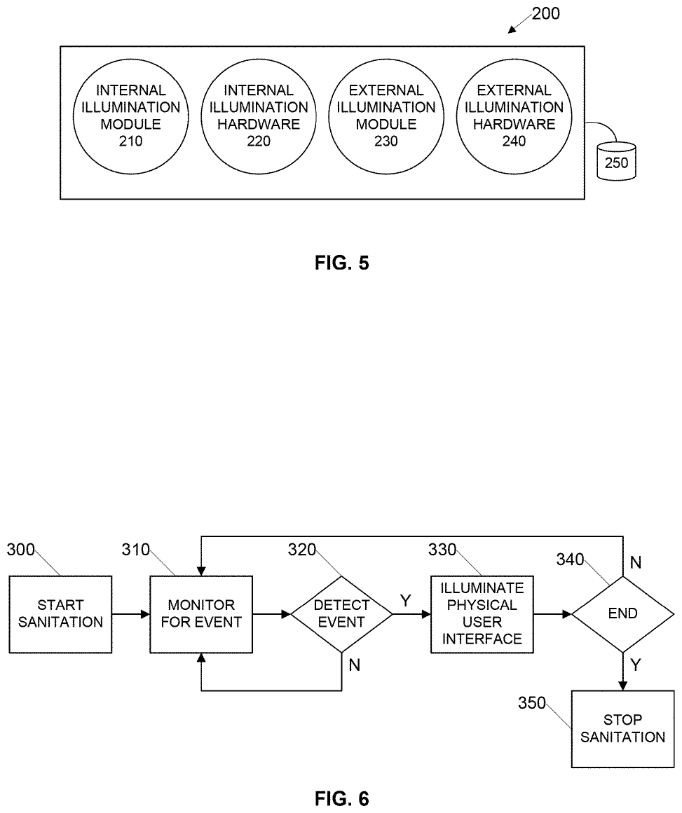

FIG. 5 is a block diagram illustrating an example touchscreen device 200 according to an embodiment of the invention. The touchscreen device 200 may be a personal touchscreen device or a public touchscreen device. In the illustrated embodiment, the touchscreen device 200 includes an internal illumination module 210, an internal illumination hardware 220, an external illumination module 230, and an external illumination hardware 240. The touchscreen device 200 may also include a data storage area 250.

The internal illumination module 210 is configured to control the internal illumination hardware 220 to cause the internal illumination hardware 220 to illuminate, from within, an external surface of a touchscreen user interface of the touchscreen device 200. Advantageously, the internal illumination hardware 220 comprises one or more illumination sources that are configured to illuminate (and thereby sanitize), from within, an external surface of a touchscreen user interface of the touchscreen device 200. The illumination comprises energy at one or more wavelengths and/or sequences of wavelengths within the electromagnetic spectrum that disrupts disease agents. For example, the illumination may be within the ultraviolet spectrum substantially between 100 nm and 400 nm. In an embodiment, the illumination may be within the short wave UV spectrum (e.g., UV-C) substantially between 200 nm and 280 nm. In an embodiment, the illumination may be at 254 nm. In an alternative embodiment, the illumination may be within the infrared spectrum.

The external illumination module 230 is configured to control the external illumination hardware 240 to cause the external illumination hardware 240 to illuminate, e.g., from above, an external surface of a touchscreen user interface of the touchscreen device 200. Advantageously, the external illumination hardware 240 comprises one or more illumination sources that are configured to illuminate (and thereby sanitize), an external surface of a touchscreen user interface of the touchscreen device 200. The illumination comprises energy at one or more wavelengths and/or sequences of wavelengths within the electromagnetic spectrum that disrupts disease agents. For example, the illumination may be within the ultraviolet spectrum substantially between 100 nm and 400 nm. In an embodiment, the illumination may be within the short wave UV spectrum (e.g., UV-C) substantially between 200 nm and 280 nm. In an embodiment, the illumination may be at 254 nm. In an alternative embodiment, the illumination may be within the infrared spectrum.

FIG. 6 is a flow diagram illustrating an example process for automated sanitation of a touchscreen device according to an embodiment of the invention. The method may be implemented by any of the previously described personal or public touchscreen devices. In one embodiment, the method is implemented by a software application that is downloaded (via an application, directly or otherwise) to or otherwise installed on the touchscreen device and configured to be executed by a processor of the touchscreen device.

Initially, in step 300 the automated sanitation process is initiated. Initiation may be automatically started upon power up or restart of the touchscreen device or it may be started in response to a command received directly via a user interface or received via a wired or wireless data communication. For example, a central controller may initiate the sanitation process on a plurality of touchscreen devices that operate as point of sale devices in a commercial operation.

Next, in step 310, the sanitation process monitors the touchscreen device for the occurrence of one or more predetermined events. A predetermined event may be the expiration of a timer that was set upon the start of the sanitation process. A predetermined event may be the conclusion of a point of sale transaction on the touchscreen device. Alternative and/or additional predetermined events may also be employed as desired or in accordance with the functionality of the touchscreen device. During monitoring if an event is not detected, as determined in step 320, the sanitation process continues to monitor for an event. If an event is detected, as determined in step 320, illumination hardware (internal or external) is controlled to cause a physical user interface to be illuminated (and thereby sanitized). After the physical user interface is illuminated, if the sanitation process is desired to continue, as determined in step 340, the process transitions back to the monitoring step 310. However, if the sanitation process is not desired to continue, as determined in step 340, the sanitation process stops as shows in step 350. Although step 340 is illustrated after the illumination step, at any time before or after any number of illumination steps the sanitation process may be ended.

Use Cases

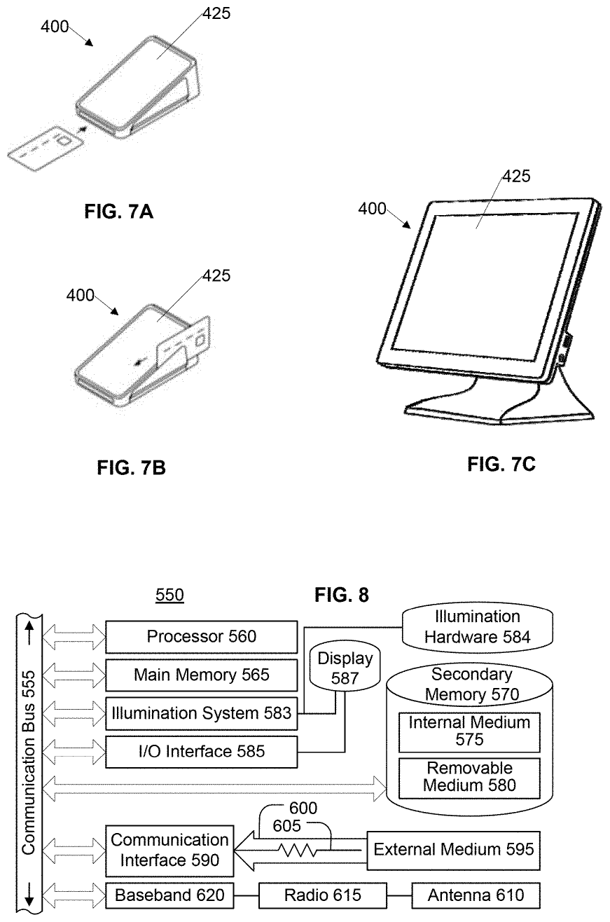

FIG. 7A-7C are block diagrams illustrating example point of sale touchscreen devices according to embodiments of the invention In the illustrated embodiment, the point of sale touchscreen devices have a touchscreen user interface 425. In one embodiment, a software application is downloaded onto the point of sale devices 400 and the application is configured to control one or more internal or external illumination sources to illuminate (and thereby sanitize) the touchscreen user interfaces 425 of the point of sale devices 400. The software application may be configured to periodically illuminate (and thereby sanitize) the touchscreen user interfaces 425 of the point of sale devices 425 and may illuminate the touchscreen with different wavelengths of energy in different sequences and may illuminate the touchscreen using other forms of energy such as sound or heat in sequence with light wave energy. The software application may also be configured to illuminate (and thereby sanitize) the touchscreen user interfaces 425 of the point of sale devices 400 upon detection of an event, for example the expiration of a timer or the completion of a point of sale transaction.

In one embodiment, a software application is downloaded onto the point of sale devices 400 and the application is configured to periodically present a message on the touchscreen user interfaces 425 of the point of sale device that prompts a user of the point of sale devices 400 to obtain a physical cleaning device (e.g., a sanitizing wipe) positioned near the point of sale device and apply the physical cleaning device to the touchscreen user interface 425s of the point of sale devices 400.

In one embodiment, an after-market string of UV-C LEDs or other energy producing devices is purchased and applied to the perimeter of a touchscreen user interfaces 425, for example a touchscreen user interfaces 425 of the point of sale devices 400. The string of UV-C LEDs or other energy producing devices may be battery powered or plugged into shore power (e.g. plugged into the point of sale devices 400 via a USB port or otherwise). The string of UV-C LEDs or other energy producing devices may constantly illuminate the surface of the touchscreen user interfaces 425 with the energy (e.g., light or sound) or may be configured to periodically illuminate the surface of the touchscreen user interfaces 425 for a predetermined amount of time, wherein the predetermined amount of time is sufficient to effectively sanitize the touchscreen user interface.

Example Device

FIG. 8 is a block diagram illustrating an example wired or wireless system 550 that may be used in connection with various embodiments described herein. For example the system 550 may be used as or in conjunction with a personal or public touchscreen device as previously described with respect to FIGS. 1A, 1B, 2A, 2B, 3A, 3B, 3C, 4 and 5. The system 550 can be a conventional personal digital assistant, smart phone, tablet computer, or any other processor enabled device that is capable of illuminating a touchscreen user interface or other physical user interface. Other processor enabled systems and/or architectures may be also used, as will be clear to those skilled in the art.

The system 550 preferably includes one or more processors, such as processor 560. Additional processors may be provided, such as an auxiliary processor to manage input/output, an auxiliary processor to perform floating point mathematical operations, a special-purpose microprocessor having an architecture suitable for fast execution of signal processing algorithms (e.g., digital signal processor), a slave processor subordinate to the main processing system (e.g., back-end processor), an additional microprocessor or controller for dual or multiple processor systems, or a coprocessor. Such auxiliary processors may be discrete processors or may be integrated with the processor 560.

The processor 560 is preferably connected to a communication bus 555. The communication bus 555 may include a data channel for facilitating information transfer between storage and other peripheral components of the system 550. The communication bus 555 further may provide a set of signals used for communication with the processor 560, including a data bus, address bus, and control bus (not shown). The communication bus 555 may comprise any standard or non-standard bus architecture such as, for example, bus architectures compliant with industry standard architecture (“ISA”), extended industry standard architecture (“EISA”), Micro Channel Architecture (“MCA”), peripheral component interconnect (“PCI”) local bus, or standards promulgated by the Institute of Electrical and Electronics Engineers (“IEEE”) including IEEE 488 general-purpose interface bus (“GPIB”), IEEE 696/S-100, and the like.

System 550 preferably includes a main memory 565 and may also include a secondary memory 570. The main memory 565 provides storage of instructions and data for programs executing on the processor 560. The main memory 565 is typically semiconductor-based memory such as dynamic random access memory (“DRAM”) and/or static random access memory (“SRAM”). Other semiconductor-based memory types include, for example, synchronous dynamic random access memory (“SDRAM”), Rambus dynamic random access memory (“RDRAM”), ferroelectric random access memory (“FRAM”), and the like, including read only memory (“ROM”).

The secondary memory 570 may optionally include a internal memory 575 and/or a removable medium 580, for example a floppy disk drive, a magnetic tape drive, a compact disc (“CD”) drive, a digital versatile disc (“DVD”) drive, etc. The removable medium 580 is read from and/or written to in a well-known manner. Removable storage medium 580 may be, for example, a floppy disk, magnetic tape, CD, DVD, SD card, etc.

The removable storage medium 580 is a non-transitory computer readable medium having stored thereon computer executable code (i.e., software) and/or data. The computer software or data stored on the removable storage medium 580 is read into the system 550 for execution by the processor 560.

In alternative embodiments, secondary memory 570 may include other similar means for allowing computer programs or other data or instructions to be loaded into the system 550. Such means may include, for example, an external storage medium 595 and an interface 570. Examples of external storage medium 595 may include an external hard disk drive or an external optical drive, or and external magneto-optical drive.

Other examples of secondary memory 570 may include semiconductor-based memory such as programmable read-only memory (“PROM”), erasable programmable read-only memory (“EPROM”), electrically erasable read-only memory (“EEPROM”), or flash memory (block oriented memory similar to EEPROM). Also included are any other removable storage media 580 and communication interface 590, which allow software and data to be transferred from an external medium 595 to the system 550.

System 550 may also include an input/output (“I/O”) interface 585. The I/O interface 585 facilitates input from and output to external devices. For example the I/O interface 585 may receive input from a keyboard or mouse and may provide output to a display 587. In an embodiment, display 587 may be a touchscreen user interface. The I/O interface 585 is capable of facilitating input from and output to various alternative types of human interface and machine interface devices alike.

System 550 may also include an illumination system 583. The illumination system 583 is configured to control illumination hardware 584 that is positioned to illuminate an external (user facing) surface of a touchscreen user interface 587. The illumination hardware 584 may be positioned to illuminate the external surface of the touchscreen user interface 587 directly or indirectly. In an embodiment, indirect illumination passes through the touchscreen user interface 587 to illuminate the external surface of the touchscreen user interface 587. The illumination hardware 584 may include one or more illumination sources such as light emitting diodes (“LEDs”) or mercury-vapor lamps. In an embodiment, illumination hardware 584 comprises one or more UV-C LEDs.

System 550 may also include a communication interface 590. The communication interface 590 allows software and data to be transferred between system 550 and external devices (e.g. printers), networks, or information sources. For example, computer software or executable code may be transferred to system 550 from a network server via communication interface 590. Examples of communication interface 590 include a modem, a network interface card (“NIC”), a wireless data card, a communications port, a PCMCIA slot and card, an infrared interface, and an IEEE 1394 fire-wire, just to name a few.

Communication interface 590 preferably implements industry promulgated protocol standards, such as Ethernet IEEE 802 standards, Fiber Channel, digital subscriber line (“DSL”), asynchronous digital subscriber line (“ADSL”), frame relay, asynchronous transfer mode (“ATM”), integrated digital services network (“ISDN”), personal communications services (“PCS”), transmission control protocol/Internet protocol (“TCP/IP”), serial line Internet protocol/point to point protocol (“SLIP/PPP”), and so on, but may also implement customized or non-standard interface protocols as well.

Software and data transferred via communication interface 590 are generally in the form of electrical communication signals 605. These signals 605 are preferably provided to communication interface 590 via a communication channel 600. In one embodiment, the communication channel 600 may be a wired or wireless network, or any variety of other communication links. Communication channel 600 carries signals 605 and can be implemented using a variety of wired or wireless communication means including wire or cable, fiber optics, conventional phone line, cellular phone link, wireless data communication link, radio frequency (“RF”) link, or infrared link, just to name a few.

Computer executable code (i.e., computer programs or software) is stored in the main memory 565 and/or the secondary memory 570. Computer programs can also be received via communication interface 590 and stored in the main memory 565 and/or the secondary memory 570. Such computer programs, when executed, enable the system 550 to perform the various functions of the present invention as previously described.

In this description, the term “computer readable medium” is used to refer to any non-transitory computer readable storage media used to provide computer executable code (e.g., software and computer programs) to the system 550. Examples of these media include main memory 565, secondary memory 570 (including internal memory 575, removable medium 580, and external storage medium 595), and any peripheral device communicatively coupled with communication interface 590 (including a network information server or other network device). These non-transitory computer readable mediums are means for providing executable code, programming instructions, and software to the system 550.

In an embodiment that is implemented using software, the software may be stored on a computer readable medium and loaded into the system 550 by way of removable medium 580, I/O interface 585, or communication interface 590. In such an embodiment, the software is loaded into the system 550 in the form of electrical communication signals 605. The software, when executed by the processor 560, preferably causes the processor 560 to perform the inventive features and functions previously described herein.

The system 550 also includes optional wireless communication components that facilitate wireless communication over a voice and over a data network. The wireless communication components comprise an antenna system 610, a radio system 615 and a baseband system 620. In the system 550, radio frequency (“RF”) signals are transmitted and received over the air by the antenna system 610 under the management of the radio system 615.

In one embodiment, the antenna system 610 may comprise one or more antennae and one or more multiplexors (not shown) that perform a switching function to provide the antenna system 610 with transmit and receive signal paths. In the receive path, received RF signals can be coupled from a multiplexor to a low noise amplifier (not shown) that amplifies the received RF signal and sends the amplified signal to the radio system 615.

In alternative embodiments, the radio system 615 may comprise one or more radios that are configured to communicate over various frequencies. In one embodiment, the radio system 615 may combine a demodulator (not shown) and modulator (not shown) in one integrated circuit (“IC”). The demodulator and modulator can also be separate components. In the incoming path, the demodulator strips away the RF carrier signal leaving a baseband receive audio signal, which is sent from the radio system 615 to the baseband system 620.

If the received signal contains audio information, then baseband system 620 decodes the signal and converts it to an analog signal. Then the signal is amplified and sent to a speaker. The baseband system 620 also receives analog audio signals from a microphone. These analog audio signals are converted to digital signals and encoded by the baseband system 620. The baseband system 620 also codes the digital signals for transmission and generates a baseband transmit audio signal that is routed to the modulator portion of the radio system 615. The modulator mixes the baseband transmit audio signal with an RF carrier signal generating an RF transmit signal that is routed to the antenna system and may pass through a power amplifier (not shown). The power amplifier amplifies the RF transmit signal and routes it to the antenna system 610 where the signal is switched to the antenna port for transmission.

The baseband system 620 is also communicatively coupled with the processor 560. The central processing unit 560 has access to data storage areas 565 and 570. The central processing unit 560 is preferably configured to execute instructions (i.e., computer programs or software) that can be stored in the memory 565 or the secondary memory 570. Computer programs can also be received from the baseband processor 610 and stored in the data storage area 565 or in secondary memory 570, or executed upon receipt. Such computer programs, when executed, enable the system 550 to perform the various functions of the present invention as previously described. For example, data storage areas 565 may include various software modules (not shown) that are executable by processor 560.

Various embodiments may also be implemented primarily in hardware using, for example, components such as application specific integrated circuits (“ASICs”), or field programmable gate arrays (“FPGAs”). Implementation of a hardware state machine capable of performing the functions described herein will also be apparent to those skilled in the relevant art. Various embodiments may also be implemented using a combination of both hardware and software.

Furthermore, those of skill in the art will appreciate that the various illustrative logical blocks, modules, circuits, and method steps described in connection with the above described figures and the embodiments disclosed herein can often be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled persons can implement the described functionality in varying ways for each particular application, but such implementation decisions should not be interpreted as causing a departure from the scope of the invention. In addition, the grouping of functions within a module, block, circuit or step is for ease of description. Specific functions or steps can be moved from one module, block or circuit to another without departing from the invention.

Moreover, the various illustrative logical blocks, modules, and methods described in connection with the embodiments disclosed herein can be implemented or performed with a general purpose processor, a digital signal processor (“DSP”), an ASIC, FPGA or other programmable logic device, discrete gate or transistor logic, discrete hardware components, or any combination thereof designed to perform the functions described herein. A general-purpose processor can be a microprocessor, but in the alternative, the processor can be any processor, controller, microcontroller, or state machine. A processor can also be implemented as a combination of computing devices, for example, a combination of a DSP and a microprocessor, a plurality of microprocessors, one or more microprocessors in conjunction with a DSP core, or any other such configuration.

Additionally, the steps of a method or algorithm described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of storage medium including a network storage medium. An exemplary storage medium can be coupled to the processor such the processor can read information from, and write information to, the storage medium. In the alternative, the storage medium can be integral to the processor. The processor and the storage medium can also reside in an ASIC.

The above description of the disclosed embodiments is provided to enable any person skilled in the art to make or use the invention. Various modifications to these embodiments will be readily apparent to those skilled in the art, and the generic principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly not limited.

1. A method comprising using at least one hardware processor to:
monitor a device having a touchscreen interface for a predetermined event to occur, and
subsequent to the occurrence of the predetermined event,
illuminate at least a portion of the touchscreen interface of the device having the touchscreen interface with one or more amounts of energy for a predetermined amount of time, wherein the one or more amounts of energy and the predetermined amount of time correspond to a desired sanitation level.

2. (canceled)

3. (canceled)

4. The method of claim 1, wherein the one or more amounts of energy comprises ultraviolet light having a wavelength in the 200 nm to 280 nm range, or light having a wavelength in the 250 nm to 260 nm range, or light having a wavelength of 254 nm.

5. (canceled)

6. (canceled)

7. (canceled)

8. The method of claim 1, wherein illuminate comprises exposing at least a portion of the touchscreen interface of the device to a sequence of different wavelengths of ultraviolet light and the order of the sequence corresponds to the desired sanitation level.

9. The method of claim 1, wherein the predetermined event is the termination of a timer and wherein the timer is automatically re-set upon termination of the timer and wherein the duration of the timer is less than one minute.

10. (canceled)

11. (canceled)

12. The method of claim 1, wherein the predetermined event is the termination of a timer and wherein the timer is automatically re-set upon termination of the timer and wherein the duration of the timer is greater than one minute.

13. (canceled)

14. The method of claim 1, wherein the predetermined event is the completion of a consumer transaction, wherein the consumer transaction is a point of sale transaction and the device having the touchscreen interface is a point of sale device.

15. (canceled)

16. (canceled)

17. (canceled)

18. A system comprising:
a non-transitory computer readable medium configured to store executable programmed modules;
a touchscreen user interface;
an illumination system configured to illuminate at least a portion of the touchscreen user interface with one or more amounts of energy;
a processor communicatively coupled with the non-transitory computer readable medium, the touchscreen user interface and the illumination system, the processor configured to control the illumination system to illuminate the at least a portion of the touchscreen user interface with the one or more amounts of energy for a predetermined amount of time, wherein the one or more amounts of energy and the predetermined amount of time correspond to a desired sanitation level.

19. (canceled)

20. (canceled)

21. The system of claim 18, wherein the ultraviolet light comprises ultraviolet light having a wavelength in the 200 nm to 280 nm range, or light having a wavelength in the 250 nm to 260 nm range, or light having a wavelength of 254 nm.

22. (canceled)

23. (canceled)

24. (canceled)

25. (canceled)

26. The system of claim 18, wherein the illumination system comprises one or more illumination sources and wherein the one or more illumination sources comprises one or more of UV-C light emitting diodes and light emitting diodes and mercury-vapor lamps and speakers.

27. (canceled)

28. The system of claim 18, wherein the illumination system comprises one or more illumination sources and wherein the one or more illumination sources is positioned to indirectly illuminate an external surface of the touchscreen user interface via an internal surface of the touchscreen user interface.

29. The system of claim 18, wherein the illumination system comprises one or more illumination sources and wherein the one or more illumination sources is positioned to directly illuminate an external surface of the touchscreen user interface.

30. The system of claim 29, further comprising one or more risers configured to position the one or more illumination sources above the external surface of the touchscreen user interface.

31. A system comprising:
a non-transitory computer readable medium configured to store executable programmed modules;
a camera system having a corresponding illumination system, the illumination system configured to illuminate an area comprising a field of view of the camera system with one or more amounts of energy;
a processor communicatively coupled with the non-transitory computer readable medium, the camera system and the illumination system, the processor configured to control the illumination system to illuminate at least a portion of an object within the area comprising the field of view of the camera system with the one or more amounts of energy for a predetermined amount of time, wherein the one or more amounts of energy and the predetermined amount of time correspond to a desired sanitation level.

32. (canceled)

33. The system of claim 31, wherein the one or more amounts of energy comprises ultraviolet light.

34. The system of claim 33, wherein the ultraviolet light comprises short-wave ultraviolet light or UV-C ultraviolet light.

35. The system of claim 33, wherein the ultraviolet light comprises light having a wavelength in the 200 nm to 280 nm range.

36. The system of claim 33, wherein the ultraviolet light comprises light having a wavelength in the 250 nm to 260 nm range.

37. The system of claim 33, wherein the ultraviolet light comprises light having a wavelength of 254 nm.

38. The system of claim 31, wherein the illumination system comprises one or more illumination sources, wherein the one or more illumination sources comprises one or more of light emitting diodes and mercury-vapor lamps and speakers.

39. (canceled)

40. The system of claim 38, wherein the one or more illumination sources comprises UV-C light emitting diodes.

41.-42. (canceled)
